(19) United States
(12) Patent Application Publication
(10) Pub. No.: US 2021/0322134 A1
(43) Pub. Date: Oct. 21, 2021

(54) CUSTOMIZED DENTAL VENEERS

(71) Applicant: SHINY SMILE VENEERS LLC, Houston, TX (US)

(72) Inventors: Amr HAMED, Houston, TX (US); Mohamed SHAMSELDEEN, Houston, TX (US)

(21) Appl. No.: 17/231,405

(22) Filed: Apr. 15, 2021

Related U.S. Application Data
(60) Provisional application No. 63/011,945, filed on Apr. 17, 2020.

(51) Int. Cl. A61C 5/20; A61C 5/30; A61K 6/891

(52) U.S. Cl. CPC A61C 5/20; A61C 2201/002; A61K 6/891; A61C 5/30

(57) ABSTRACT

Described is a customized removable cosmetic or aesthetic dental veneer that secures onto a row of teeth of a patient. The dental veneer may include a pair of rear portions that secure it onto the row of teeth, each rear portion positioned at a distal end of the veneer and having: buccal, occlusal and lingual sides for one or more rear tooth, and a front portion between the pair of rear portions that covers the buccal and occlusal surfaces of the teeth between the pair of rear portions but is free of a side that covers a majority of the lingual surface of any tooth of the row of teeth between the pair of rear portions. A slot and apertures may also be provided for occlusal portions of the rear teeth. Also described is a veneer without such rear portions, which may be used with an adhesive.

### CROSS-REFERENCE TO RELATED APPLICATION(S)

This non-provisional patent application claims priority to U.S. Provisional Patent Application 63/011,945 filed Apr. 17, 2020, the entire contents of which are incorporated herein by reference.

### FIELD OF THE INVENTION

The present invention is in the field of customizable dental veneer for improved aesthetic appearance, more particularly, a dental veneer that may be ordered online and custom made and that may feel more natural for the wearer/patient.

### BACKGROUND OF THE DISCLOSURE

A person's teeth can often have an important role in the overall visual impression the person creates in other people. Dental veneers can enhance the aesthetic appearance of teeth by hiding stains, misalignments and other imperfections and by providing a unified, cleaner look to a row of teeth.

Customizable, removable dental veneers are known. However, they often create pressure points or feel heavy or cumbersome in the mouth. They can impair or adversely affect the flow of saliva and thus feel onerous and unnatural. Also, they can come between the tongue and the front teeth during speech and thus interfere with the production of particular phonemes, or at least make take some getting used to. They can feel heavy and can interfere with the natural flow of saliva and fluids and may make chewing more difficult, particularly if they cover most or all of the chewing surfaces of the molars.

Various types of dental veneers are known, for example, U.S. Pat. Nos. 7,357,637, 8,459,296, and 9,901,429 and U.S. Patent Application Publication 2017/0196657A1. Additional known art in this field includes references with the following patent/patent application publication numbers: U.S. Pat. No. 6,884,073B2, US 2019/0201165A1, US 2004/0096805A1, US 3,565,387, US 2020/0060797A1, U.S. Pat. No. 10,383,701B2, US 2018/0280116A1, US 2019/0298489A1, US 2011/0081630A1, US 2014/0113250A1, US 2018/0132973A1, US 2005/0227204A1, US2017/0196657A1, EP 0014860A1, WO 2020/033532A1, and CN110063801A. Each of these patents and patent application publication documents is incorporated in full herein by reference.

### SUMMARY OF THE DISCLOSURE

Described is a removable dental veneer that secures onto a row of teeth of a patient. The dental veneer may include a pair of rear portions configured to secure the dental veneer onto the row of teeth, each rear portion positioned at a distal end of the veneer and having:

a buccal side configured to cover a buccal surface of at least one rear tooth, an occlusal side configured to cover at least a majority of an occlusal surface of the at least one rear tooth, and a lingual side configured to cover at least a majority of a lingual surface of the at least one rear tooth; and

a front portion positioned between the pair of rear portions and comprising:

a buccal side configured to cover a buccal surface of each tooth of the row of teeth between the pair of rear portions, and an occlusal side configured to cover an occlusal surface of each tooth of the row of teeth between the pair of rear portions,

wherein the front portion is free of a surface that covers a majority of the lingual surface of any tooth of the row of teeth between the pair of rear portions.

In such a dental veneer, each rear portion may cover at least two rearmost teeth or three rearmost teeth.

Such a dental veneer may also be paired with a corresponding removable dental veneer that secures onto an opposing row of the patient's teeth, the corresponding dental veneer having:

a corresponding pair of rear portions that secure the corresponding dental veneer onto the opposing row of teeth, each rear portion of the corresponding pair positioned at a distal end of the corresponding dental veneer and comprising:

a buccal side configured to cover substantially all of a buccal surface of at least one rear tooth, an occlusal side configured to cover substantially all of an occlusal surface of the at least one rear tooth, and a lingual side configured to cover substantially all of a lingual surface of the at least one rear tooth; and

a corresponding front portion positioned between the corresponding pair of rear portions and comprising:

a buccal side configured to cover substantially all of a buccal surface of each tooth of the opposing row of teeth between the corresponding pair of rear portions, and an occlusal side configured to cover substantially all of an occlusal surface of each tooth of the opposing row of teeth between the corresponding pair of rear portions,

wherein the corresponding front portion is free of a surface that covers a majority of the lingual surface of any tooth of the opposing row of teeth between the corresponding pair of rear portions,

wherein in the dental veneer, the buccal side of the front portion covers substantially all of the buccal surface of each tooth of the row of teeth between the pair of rear portions, and the occlusal side covers substantially all of the occlusal surface of each tooth of the row of teeth between the pair of rear portions.

In such a dental veneer, the pair of rear portions may retain the dental veneer on the row of teeth by elastic deformation of the pair of rear portions and free of an adhesive compound.

In such dental veneer, each rear portion may cover an entirety of the occlusal surface of the at least one rear tooth.

In such a dental veneer, the front portion may include a lingual ridge that extends from the occlusal side and is positioned over a lingual portion of the lingual surfaces immediately adjacent the occlusal surfaces of the row of teeth between the rear portions, such that the lingual ridge covers less than an tenth of the visible lingual surfaces of the row of teeth between the rear portions.

Such a dental veneer may be a customized dental veneer fitted for the patient's row of teeth.

Such a dental veneer may be translucent or semi-transparent.

Such a dental veneer may be made substantially entirely of polyethylene terephthalate or PETG.

Also described is a removable dental veneer that secures onto a row of teeth of a patient, in which a pair of rear portions secures the dental veneer onto the row of teeth, each rear portion positioned at a distal end of the dental veneer and including:

a buccal side that covers at least a majority of the buccal surface of at least one rear tooth of the row of teeth,

a lingual side that covers at least a majority of a lingual surface of the at least one rear tooth,

an occlusal side that covers a periphery of an occlusal surface of the at least one rear tooth and defines a slot extending from a distal end of the row of teeth over the occlusal surface of the at least one rear tooth, and

the occlusal side having a ridge adjacent the slot and positioned adjacent a second surface of the at least one rear tooth, the second surface being a mesial surface opposite the distal end of the row of teeth, the ridge connecting the buccal side and the lingual side,

such that the occlusal side covers a periphery of an occlusal surface of an additional rear tooth immediately adjacent the at least one rear tooth, the occlusal side defining an aperture exposing at least a majority of the occlusal surface of the additional rear tooth; and

the veneer also having a front portion positioned between the pair of rear portions and including:

a buccal side that covers a buccal surface of each tooth of the row of teeth between the pair of rear portions, and an occlusal side that covers an occlusal surface of each tooth of the row of teeth between the pair of rear portions,

such that the front portion is free of a surface that covers a majority of the lingual surface of any tooth of the row of teeth between the pair of rear portions.

Such a dental veneer's aperture may expose most of the occlusal surface of the additional tooth.

In such a dental veneer, an entirety of the perimeter of the slot may be curvilinear and free of acute angles.

In such a dental veneer, the slot may be positioned to expose at least a majority of the occlusal surface of the at least one tooth.

In such a dental veneer, the pair of rear portions may retain the dental veneer on the row of teeth by elastic deformation of the pair of rear portions and free of an adhesive compound.

In such a dental veneer, the front portion may include:

a thin lingual ridge extending from the occlusal side and positioned over a periphery of the lingual surfaces of the row of teeth immediately adjacent the occlusal surfaces of the row of teeth between the pair of rear portions.

Such a dental veneer may be a customized dental veneer fitted for the patient's row of teeth.

The dental veneer of claim 10, wherein the dental appliance is a customized

dental veneer fitted for the patient's row of teeth.

In such a dental veneer, the slot may be sized and positioned to reveal an entirety of the occlusal surface of the at least one rear tooth except for a thin occlusal ridge at the buccal, lingual and mesial periphery of the occlusal surface.

Such a dental veneer may be translucent or semi-transparent. For example, it may be made substantially entirely of at least one of polyethylene terephthalate or PETG.

Also described is a removable dental veneer that secures onto a row of teeth of a patient, including a pair of rear portions configured to secure the dental veneer onto the row of teeth, each rear portion positioned at a distal end of the veneer and including:

a buccal side that covers at least a majority of a buccal surface of at least one rear tooth, a lingual side that covers at least a majority of a lingual surface of the at least one rear tooth, and an occlusal side that covers at least a majority of an occlusal surface of the at least one rear tooth, and the occlusal side defining a first aperture positioned to expose a central portion of the occlusal surface of the at least one tooth; and

a front portion positioned between the pair of rear portions and including:

a buccal side that covers a buccal surface of each tooth of the row of teeth between the pair of rear portions, and an occlusal side that covers an occlusal surface of each tooth of the row of teeth between the pair of rear portions.

In such a dental veneer the front portion may also include a lingual side covers at least a majority of the lingual surface of each tooth of the row of teeth between the pair of rear portions. Or, the front portion may further include a slim lingual ridge that covers a portion of a lingual surface of each tooth of the row of teeth between the pair of rear portions, such that the slim lingual ridge covers no more than a tenth of the lingual surface of each tooth of the row of teeth between the pair of rear portions. In the alternative, there may be no lingual side or ridge whatsoever, according to a further aspect of the disclosure.

In such a dental veneer, the first aperture may be sized and positioned to expose only a central portion of the occlusal surface of the at least one tooth and the occlusal side is configured to cover all remaining portions of the at least one tooth.

In such a dental veneer, the at least one tooth may include a rearmost tooth and a second tooth adjacent the rearmost tooth, and the occlusal side of the rear portion may then define a second aperture positioned to expose a central portion of the occlusal surface of the second tooth, the second aperture being of the same size and shape as the first aperture, such that the occlusal side of the rear portion covers all remaining portions of the occlusal surface of the rearmost tooth and the occlusal surface of the second tooth.

In such a dental veneer, the first aperture may be substantially round. Also, the first aperture may have curvilinear edges but may not be round or substantially round.

In such a dental veneer, the at least one tooth may in fact include a rearmost tooth, a second tooth adjacent the rearmost tooth, and a third tooth adjacent the second tooth, and the occlusal side of the rear portion may a second aperture positioned to expose a central portion of the occlusal surface of the second tooth and a third aperture positioned to expose, respectively, central portions of the occlusal surfaces of the second and third teeth, the second aperture and the third aperture being substantially of the same size and shape as the first aperture, such that the occlusal side of the rear portion covers all remaining portions of the occlusal surfaces of the rearmost tooth, the second tooth, and the third tooth. For example, each of the first, second and third apertures may exposes a minority of the occlusal surface of the respective tooth while a majority of the respective tooth is covered by the occlusal side of the veneer.

Such a dental veneer may be a customized dental veneer fitted for the patient's row of teeth.

Such a dental veneer may be translucent or semi-transparent. For example, it may be made substantially entirely of polyethylene terephthalate or PETG.

Also described is a dental veneer that secures onto a row of teeth of a patient, and including:

a buccal side that covers a buccal surface of each tooth of the row of teeth; and

an occlusal side that covers an occlusal surface of each tooth of the row of teeth, wherein the dental veneer is free of a surface that covers a majority of the lingual surface of any tooth of the row of teeth.

Such a dental veneer may further include a slim lingual ridge that extends from the occlusal side and is positioned over a peripheral portion of the lingual surface of the row of teeth immediately adjacent the occlusal surfaces of the row of teeth.

In such a dental veneer, the buccal side may cover substantially all of the buccal surface of each tooth of the row of teeth, and the occlusal side covers substantially all of the occlusal surface of each tooth of the row of teeth.

Such a dental veneer may be a customized dental veneer fitted for the patient's row of teeth and secured to the row of teeth by an adhesive compound. For example, the dental veneer may be translucent or semi-transparent. By way of illustrative example, such a dental veneer may be made substantially entirely of polyethylene terephthalate or PETG.

Properties or features described or illustrated with respect to one aspect of the disclosure, or recited with respect to one claim below, may be combined with, or substituted for, one or more properties or features described or illustrated with respect to another aspect of the disclosure, or recited with respect to another claim or portion thereof. Contemplated are many such additional combinations of features, and many such substitutions of features, not explicitly noted or illustrated.

### BRIEF DESCRIPTION OF THE DRAWINGS

FIGS. 1-10 illustrate an example of a dental veneer according to an aspect of the present disclosure.

FIGS. 11, 12 and 15 illustrate an example of a second aspect of the disclosure with occlusal slots in the veneer corresponding to hind teeth, showing this veneer with a mold that may be used in the manufacturing process.

FIGS. 13 and 14 illustrate an example of the second aspect of the disclosure shown in FIGS. 11, 12 and 15 but without the molds.

FIGS. 16-18 illustrate an example of third aspect of the disclosure, with occlusal apertures or holes in the veneer corresponding to some or all teeth, for example, hind teeth.

FIGS. 19-21 illustrate an example of a fourth aspect of the disclosure with a veneer that includes a covering only one side of the teeth, with a mold.

FIGS. 22 and 23 illustrate an example of the fourth aspect of the disclosure shown in FIGS. 19-21 without the mold.

The Drawings illustrate examples of aspects of the invention. Other features and advantages of the present invention will become apparent from the following description of the invention, and/or from the combination of one or more of the figures and the textual description herein, or from portions thereof.

### DETAILED DESCRIPTION OF THE EMBODIMENTS

According to an aspect of the disclosure, dental veneer 40 may be a removable elective dental appliance that is designed to enhance the appearance of the user's teeth is provided. The veneer 40 may be put in, worn, and removed by the user as he/she wishes. The veneer may be securely retained by slight elastic deformation of parts of the veneer, such as the rear parts that sit on the molars described below, caused by gentle pressure from parts of the teeth.

The veneer may comprise, or may be made entirely of, translucent PET (polyethylene terephthalate) or PETG. However, it will be understood that transparent or semi-transparent, white or off-white materials and other colors or combinations of colors may also be used. Also, the veneer may be made of a combination of different plastics or other materials or of materials of different color, of different color gradations, of different opacity, or of varying scale of opacity.

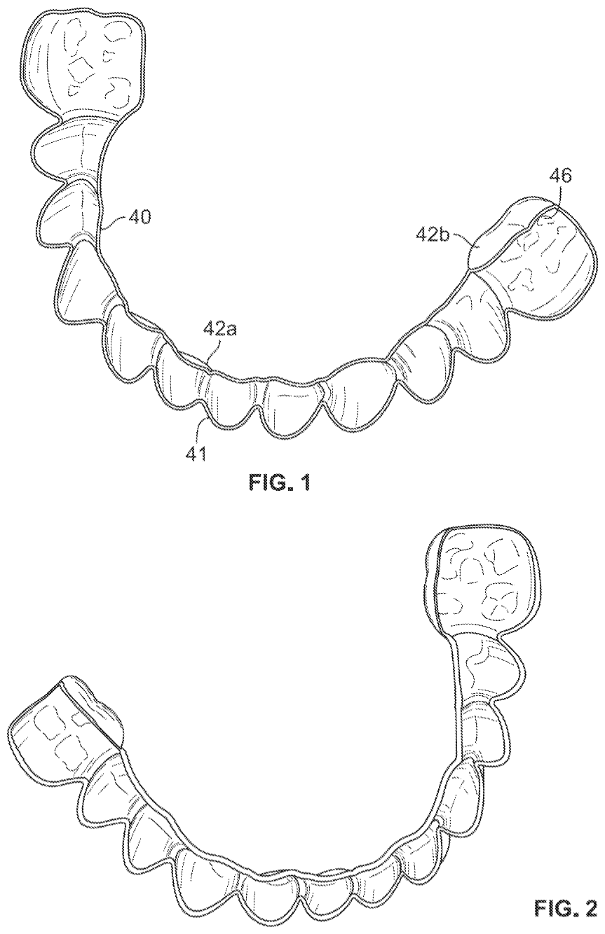

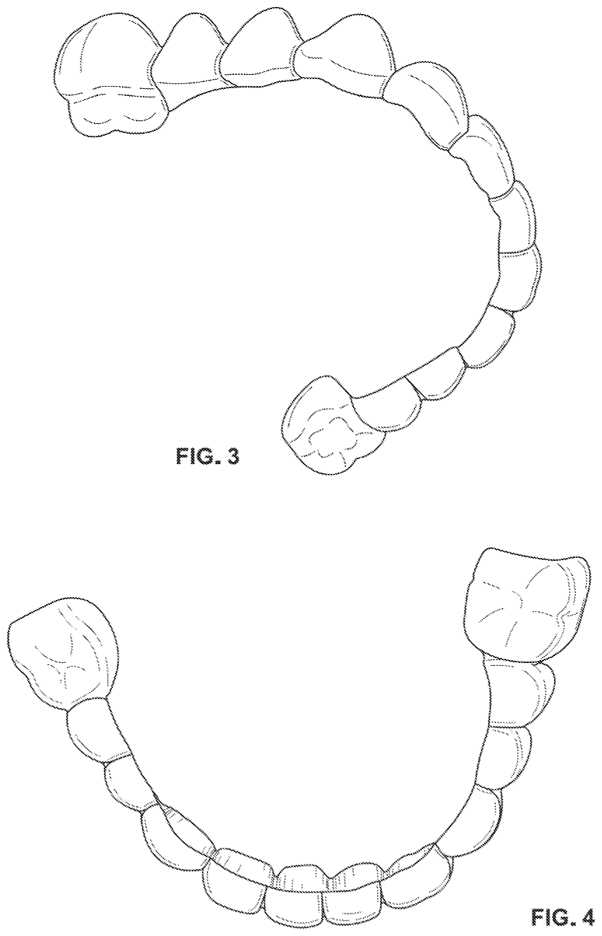

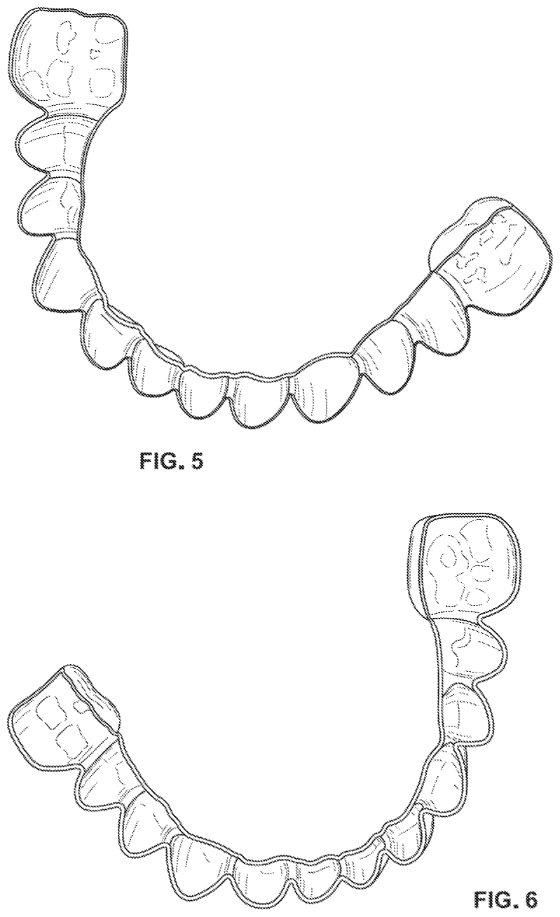

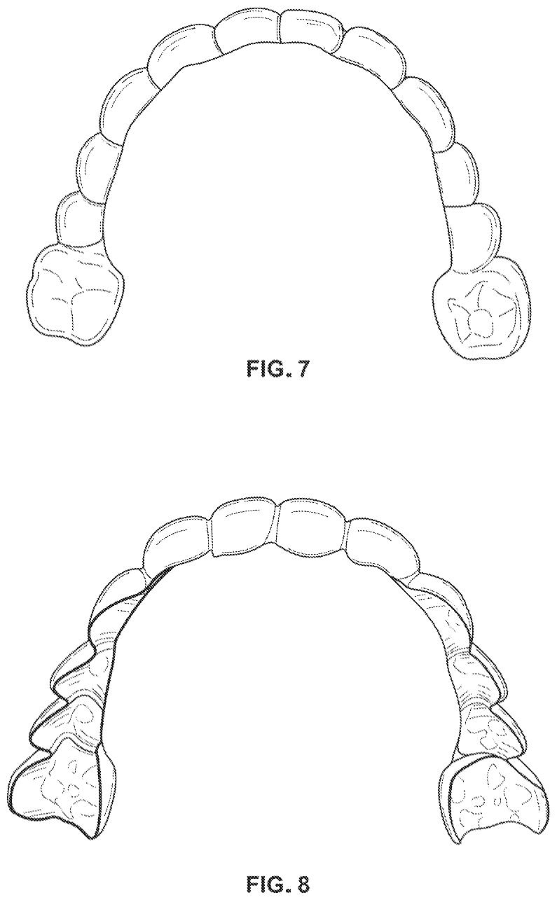

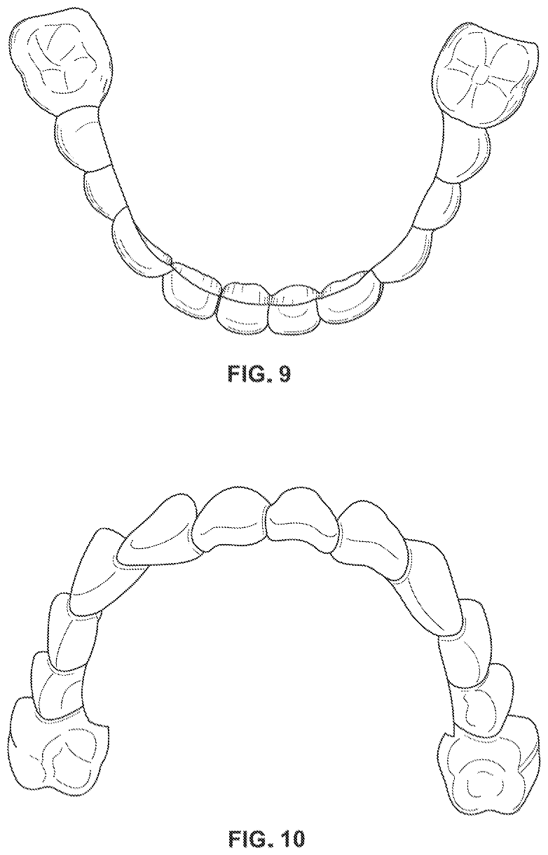

According to an aspect of the disclosure as illustrated in FIGS. 1-10, the veneer 40 may have a front portion that has a buccal side 41 that covers exclusively the outer surfaces on the buccal (cheek) side of the user's teeth. “Cover” in this context may mean that the veneer achieves an aesthetic goal that no substantial portion of the buccal side of the teeth between the rear portions is visible to an observer in the course of the wearer performing ordinary life activities. In this way, without a substantial lingual side for the veneer, a lighter, less obtrusive and/or less cumbersome dental appliance may be achieved. Eating and other functions may be interfered with to a lesser degree and natural flow of saliva may be maintained to an improved degree compared with traditional removable veneers. A more natural feel and improved speech may be achieved while the veneers are worn because when the lingual side of the teeth is totally, substantially totally, or at least mostly, exposed and free of being covered by the veneer, there is a reduced chance of the tongue coming into contact with the veneer.

As illustrated in FIGS. 1 and 2, according to a first aspect of the present disclosure, a dental veneer may cover also the biting (occlusal) surfaces of the teeth. It may also include a small peripheral lingual ridge 42a on the lingual side of the teeth extending downward (or upward in the case of veneer for the upper row of teeth) from the biting surfaces. This lingual ridge may have a role in retention in place on the teeth.

In addition, the veneer may include a pair of rear portions, with a rear portion at each distal end of the veneer that corresponds to and surrounds the rearmost teeth, in individuals who have molars this would mean the molars, at each end of the arch: on the cheek side, the biting surface, and the lingual side of the rearmost teeth so as to retain the veneer on the teeth. The rearmost tooth/teeth may mean the wisdom tooth or the adjacent molar, or may mean the wisdom tooth on one distal end of the veneer and the adjacent molar at the second distal end of the veneer, for example, if the patient lacks a wisdom tooth at the second side. The veneer may also be secured onto teeth other than molars, for example, if the patient lacks molars on one or both sides of the row of teeth. All or substantially all, or at least a majority, of the lingual side of the teeth between the rear portions that cover the rearmost teeth may remain exposed free of being covered by the veneer. All, nearly all, or substantially all may mean that all or virtually all of the respective exposed or visible surface of the tooth is covered from the perspective of a person viewing the patient. Substantially all, or substantially entirely, may mean all or entirely from a practical point of view to provide an acceptable hold of the veneer and/or an aesthetic point of view. A majority of a surface of a tooth may mean most of the exposed or visible surface from an aesthetic perspective when a viewer is looking at the wearer/patient. While illustrating the veneer for only one row of teeth, it will be understood that a veneer for the corresponding upper or lower teeth may also be provided.

The rear portion that secures onto one or more of the rearmost teeth, for example, the two adjacent molars, may be provided at each distal end of the veneer. Each rear portion of such a pair of rear portions may include a molar cup 46 that snaps on (latches on by elastic deformation) to the rear molar or molars at each end of the row of teeth. This may obviate the need for the use of an adhesive compound to secure the veneer onto the teeth. In this way, the veneer may be conveniently removable and repositionable by the patient throughout the day as needed or desired. At the same time, a less heavy and less obtrusive feel may be provided for most of the teeth without a lingual side wall.

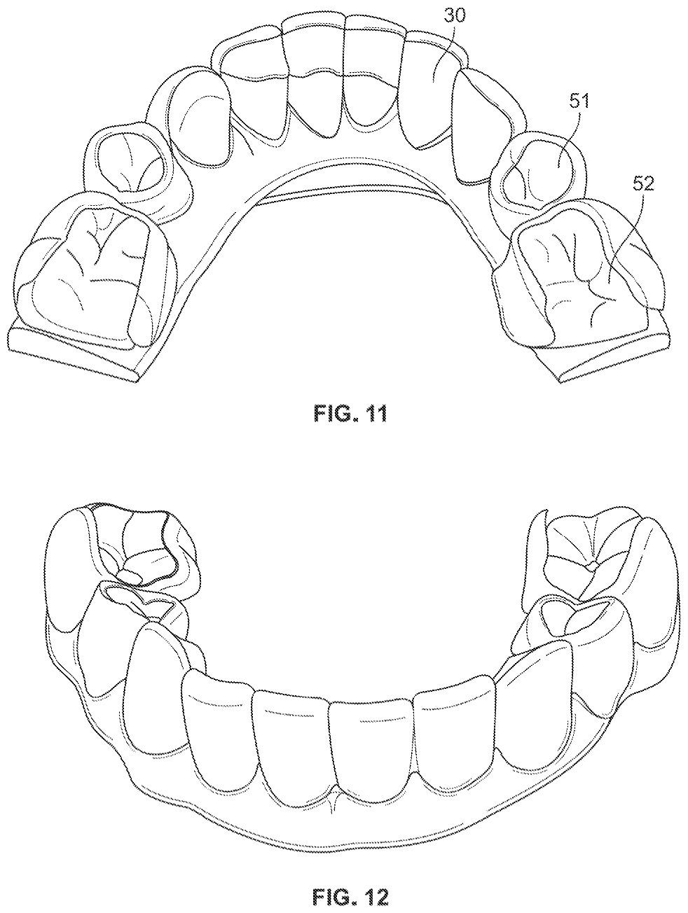

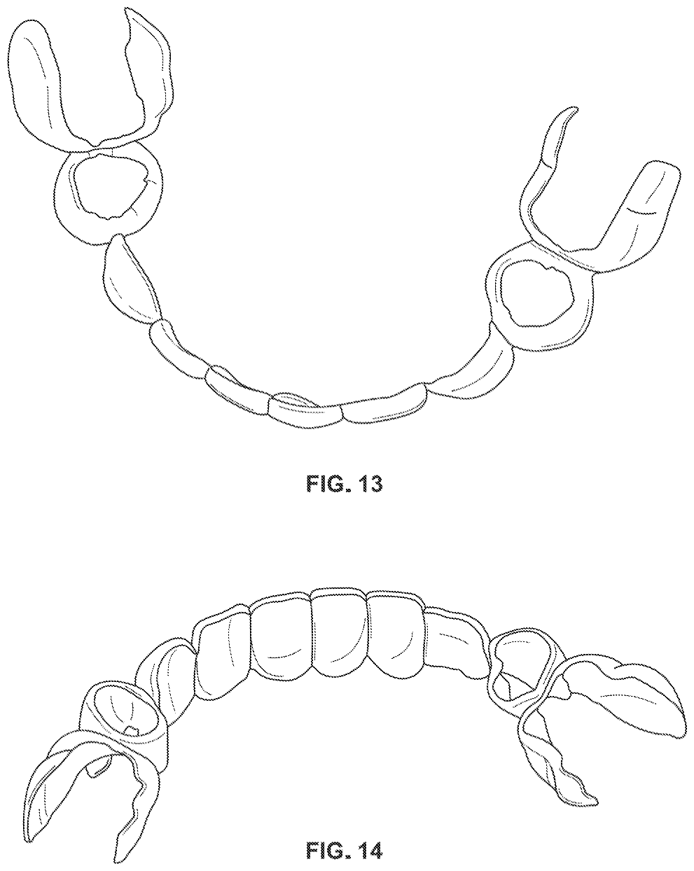

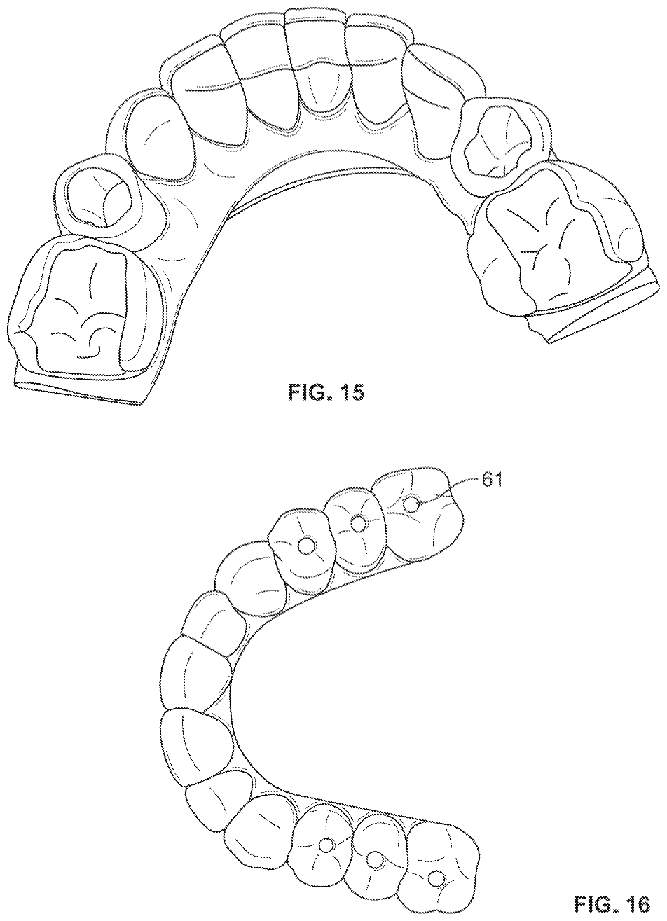

The securing on the veneer by virtue of these rear portions may be similar in concept according to the second aspect of the disclosure shown in FIGS. 11-15. Also, the second aspect of the invention as shown in FIGS. 11-15 may be similar to the first aspect of the disclosure shown in FIGS. 1-10 in that the front part of the veneer, the portion between the rear portions at each distal end, may cover the buccal and occlusal surfaces of the teeth but not most, or not any, of the lingual sides.

The front portion may include a small, peripheral occlusal ridge that covers a small peripheral occlusal surface of the front teeth immediately adjacent the occlusal surfaces. In this way, with the slot and hole, a lighter weight and improved feel may be achieved. According to a further aspect of this second aspect of the disclosure shown in FIGS. 11-15, although the front portion of the veneer illustrated in FIGS. 11-15 lacks a lingual surface, however, the front portion may also include a lingual side that covers most, all, or substantially all of the lingual surfaces of the row of teeth between the between rear portions so that other aims of the veneer are achieved.

According to the aspect of the disclosure illustrated in FIGS. 11-15, a rear slot or slit 52 that extends from the distal end of the dental veneer over a central or near central portion of the occlusal surface of the rearmost tooth, or over more than one rearmost tooth, is provided. While described herein as central, according to an aspect of the disclosure slot 52 need not be positioned at a precise center of the veneer or center of the occlusal surface of the rearmost tooth measured from the buccal side to the lingual side. Rather, according to an aspect of the disclosure central or center in the context of the slot may mean a non-peripheral occlusal portion, or an area of the occlusal portion that is not immediately adjacent the buccal or lingual surfaces. In the alternative, slot may be positioned substantially centrally with respect to the buccal and lingual sides.

In addition, corresponding to a second tooth, immediately adjacent the rearmost tooth over which is the above-described slot, the rear portion may define an aperture 51 to expose a central portion of an occlusal surface of the second tooth. Aperture 51 may be positioned centrally or substantially centrally, as that term is described above. Aperture 51 may expose all, or nearly all, of the occlusal surface of the second to rearmost tooth. However, aperture 51 may be defined by thin peripheral occlusal ridge of the veneer that is positioned to cover a peripheral occlusal area of the second rearmost tooth. According to a further aspect of the disclosure, aperture 51 may have a substantially circular or near circular shape such that the peripheral areas of the second to rearmost tooth remain unexposed by the veneer.

Slot 52 and the aperture 51 may be separated by a ridge of the veneer that is above an interdental region between the rearmost and second rearmost teeth is also illustrated in FIGS. 11-15. This ridge may be formed as a bridge between the buccal and lingual walls of the veneer. This ridge may be also extend slightly over a peripheral region of the occlusal surfaces of the rearmost and next to rearmost teeth. In the alternative, or in addition, this ridge may extend slightly downward (or slightly upward in the case of a veneer for the upper row of teeth) to cover a small portion of a first interdental surface of the rearmost tooth and a small portion of the second interdental surface of the next to rearmost tooth, the first and second interdental surfaces facing each other. In addition or in the alternative, the ridge may remain just at the level of the occlusal surfaces of the teeth without extending at all into the interdental gap between these teeth.

Such a slot 52 and aperture 51 may have the advantages of improved saliva flow and digestion, improved feel during chewing, reduced wear and tear on the veneer resulting from grinding of the molars, and a lighter weight and a more natural feel for the wearer of the veneer.

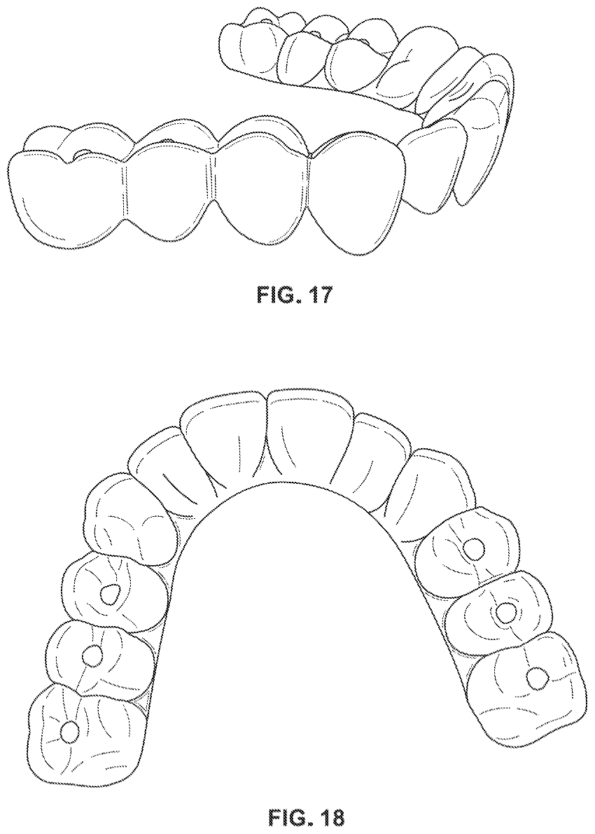

As illustrated in FIGS. 16-18, according to a third aspect of the disclosure, occlusal holes or apertures 61 may be provided on the veneer corresponding to the biting surface of one or more teeth, for example, at the molars and/or the premolars, to further lighten the weight and to improve the feel of the veneer. In other respects, aside from the occlusal holes or apertures 61, this third aspect of the disclosure may be quite similar to the first aspect of the disclosure illustrated in FIGS. 1-10, that is, a lingual side wall be absent for the portion of the veneer between the rear portion, with the possible exception of a thin lingual ridge 42 as described above, which may be present. According to a further version of the disclosure as shown in FIGS. 16-18, this third aspect of the disclosure may be different from the first aspect of the disclosure also for the reason that the front portion of this veneer between the rear portions may include a lingual side 42 that covers all, nearly all, or most of the exposed portion of the lingual surfaces of the front teeth of the row of teeth.

Apertures or holes 61 may be approximately 2-3 mm in diameter. The holes may be uniform for the occlusal side of each tooth. The holes may be substantially circular. Substantially circular may mean that the holes convey a visual impression of circularity when the veneer is in the mouth or when held by a casual observer, or that the holes are created by a standard instrument or machine implement used in the field to create circular apertures. The holes may be substantially oval or have a curvilinear perimeter. Substantially oval may mean that the holes convey a visual impression of being oval when the veneer is in the mouth or when held by a casual observer, or that the holes are created by a standard instrument or machine implement used in the field to create oval apertures. According to a further aspect of the disclosure illustrated in FIGS. 11-15, aperture 51 with respect to its shape may be similar to the holes shown in FIGS. 16-18. It will be understood that according to an aspect of the disclosure, the holes may be slightly larger or smaller than the holes shown in FIGS. 16-18. While shown as uniform in size and shape, according to a further aspect of the disclosure a hole may be sized in proportion to the overall size of the teeth over which it is positioned.

The holes may be located exactly in the center of back teeth, such as molars and premolars in the case of patients who have molars and premolars. According to a further aspect of the disclosure, the holes may be approximately at or near the center, or at least that the hole is not only near the periphery. According to a further aspect of the disclosure, the holes may be provided at portions of the veneer corresponding to teeth additional to the molars and premolars. In this way, fewer than three holes or more than three holes may be provided at each end of the veneer.

According to an aspect of the disclosure, as illustrated in FIGS. 16-18, the holes 61 may be positioned only on the occlusal surface and not to reach any other surface. According to a further aspect of the disclosure, the holes may be only oval or only circular in shape, or may be either circular or oval in shape. According to a further aspect of the disclosure, the holes may have only curvilinear perimeters but not be necessarily be circular, substantially circular, oval or substantially oval, so that by being free of sharp or acute angles at any point, they are more resistant to wear and tear. According to a further aspect of the disclosure, the holes may have other shapes and/or holes over some teeth may be oval shape while holes over other teeth may have another shape.

Some functions that the occlusal holes may provide include making the appliance lighter, facilitating natural escape and flow for saliva and fluid build up inside the veneers, and affording additional resilience and flexibility to back teeth to engage undercuts easily. However, it will be understood that not all of these functions may be provided by all aspects of the disclosure. Features and structures discussed or illustrated with respect to one aspect of the disclosure, for example, the veneer shown in FIGS. 16-18 may be combined with, or substituted for, other features or structures, or omitted entirely.

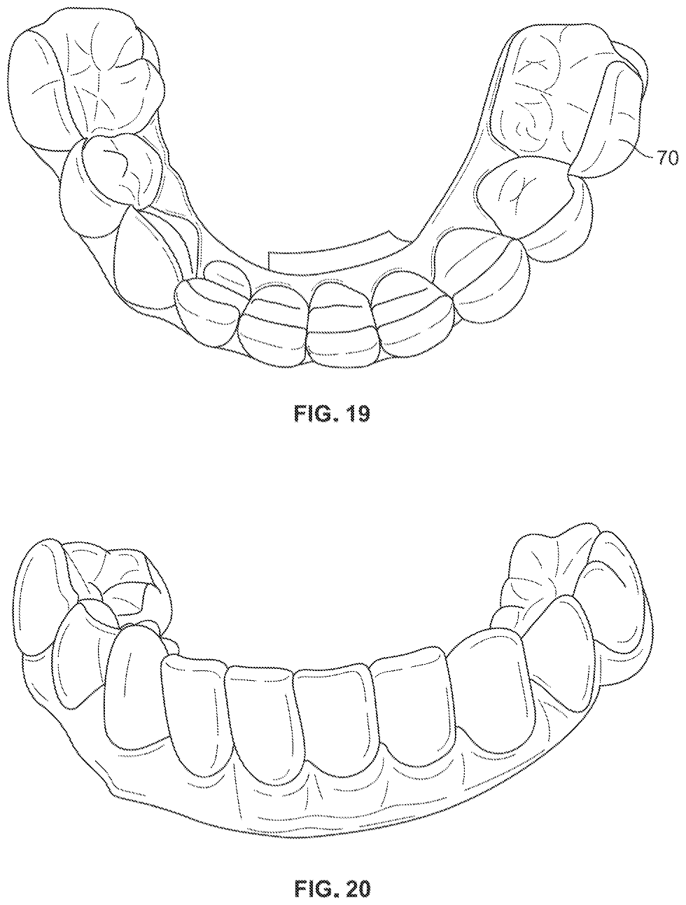

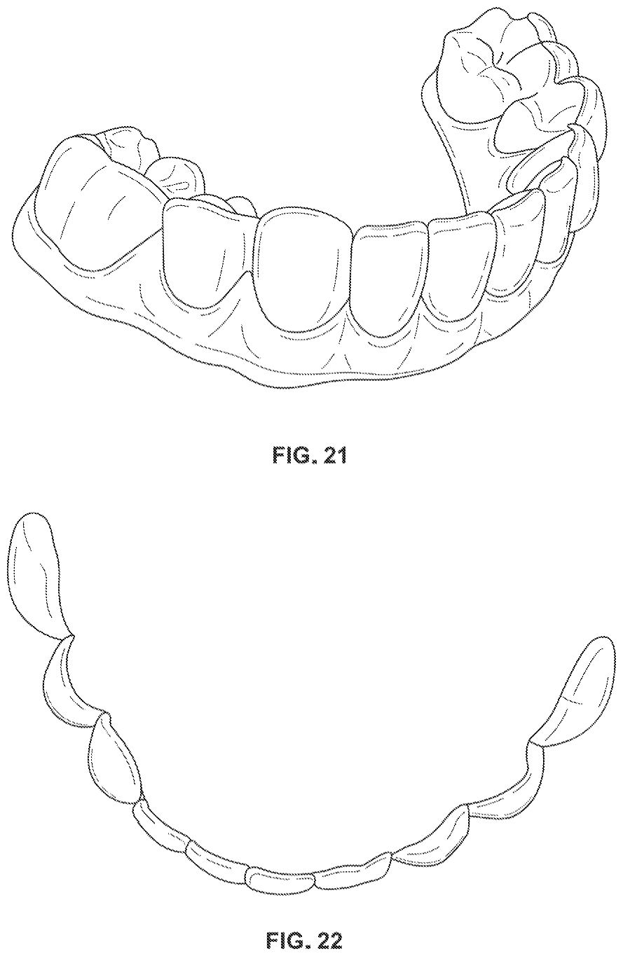

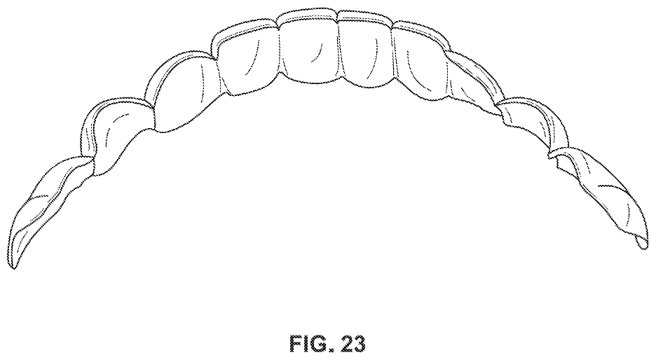

According to a further aspect of the disclosure, an extended finish line for the palatal and lingual sides may be provided for the veneer shown in FIGS. 16-18. However, such a finish line is also contemplated for the versions shown in FIGS. 1-10, FIGS. 11-15, and/or FIGS. 19-23. The extended finish lines may cover part of the gum line or part of the gums adjacent the teeth so as to provide a more comprehensive aesthetic improvement for patients who need or desire an enhanced appearance for those additional regions.

One straight finish line may be provided that does not extend between teeth. This may be located on the palatal and lingual sides of the dental appliance. Such an implementation need not extend to labial or buccal surfaces of the appliance. It may cover teeth and parts of the embrasures between teeth.

Some functions of the extended finish line may be that the appliance may be sturdier, increased or improved retention and stability of the appliance may be achieved, the appliance may be less prone to fractures during insertion and removal, a more comfortable feel of customer's gingiva may be obtained, it may be make it more possible to extend the appliance over free end saddle areas, it may make it more possible to bridge the appliance between teeth in case of multiple missing teeth. However, it will be understood that not all of these functions may be provided or obtained by all aspects of the disclosure with or without the extended finish line.

Illustrated in FIGS. 19-23 is a fourth aspect of the disclosure in which the veneer is provided only on the cheek side of the teeth.

According to the aspect of the disclosure illustrated in FIGS. 19-23, no lingual side of the veneer need be present. The veneer may cover occlusal surfaces of the teeth.

According to a further aspect of the disclosure not illustrated in FIGS. 19-23, a slim lingual side ridge may also extend from the occlusal surface. However, this is not illustrated and not necessary for the aspect of the disclosure illustrated in FIGS. 19-23.

With respect to the veneer shown in FIGS. 19-23, an adhesive may be needed to secure the veneer to the teeth, in which case the appliance may be considered a permanent appliance (not a removable appliance), so that the customer/patient may leave it in overnight, during eating, and the like.

An adhesive compound, for example, a commercially available over-the-counter dental appliance adhesive, may be applied by the customer himself or herself and the veneer thereafter inserted onto the row of teeth. In this way, the veneer may still be removable by the customer by exerting a focused force to remove the veneer. Adhesives for such use with dental appliances are well known. Veneers illustrated herein in FIGS. 1-18 may also be used with adhesives. However, according to an aspect of the disclosure, adhesives need not be used with those veneers in the removable mode.

As will be understood, such customizable veneers may be fashioned in an office visit or online. A customer may place an order online and receive a kit that the customer may use at home to create a dental impression, which the customer may then return by mail for the veneer to be manufactured. According to an aspect of the disclosure, the dental veneer may be translucent or semi-transparent. Translucent or semi-transparent may mean that from a practical aesthetic point of view, when the patient is viewed, some imperfections of the patient's teeth are not visible or at least that the visibility of some imperfections is substantially hindered by the veneer. Such a veneer may provide a natural look for the teeth, and/or the presence of the veneer may be difficult for a casual observer to notice, but at the same time the veneer may achieve an enhanced aesthetic appearance as stains, discolorations, yellowing, chipping of teeth, fillings, crowns and/or other dental work, misalignment of teeth and other imperfections may be hidden or partially hidden, or at least their visibility may be reduced. According to an aspect of the disclosure, the veneer may be available in a range of translucencies depending on the patient's teeth imperfections and/or how much of the imperfections the patient wishes to hide, and/or may be available in various shades, according to preference, for example, om1, a1, a2 (om1 is the brightest and a2 is less white), or a color or tint depending on the needs and preferences of the patient.

The customer may desire any specific shape, and enter this preference in the notes, or the best shape may be automatically selected appropriate for the smile and contours of the teeth of the customer

After the order is placed, an impression kit with instructions may be shipped or otherwise given to the customer, so that customized mold 30 of the customer's teeth may be obtained. The mold 30 may vary according to the type of dental veneer being made and the size and shape of the particular patient's teeth. Then, the mold 30 may be 3d scanned using a 3 shape dental scanner to have a digital picture of the mold. The 3d dental scan is then modified and shaped digitally according to the customer needs. The scan may then be then 3d printed using a 3d dental printer so than an exact duplicate of the customer's teeth may be obtained. A 3d printed dental cast may then be shaped and modified by wax. The wax may then be modified model and then duplicated. Using dental plastic colored sheets (om1, a1, a2), for example, Erokdur obtained from Erkodent company made from PETG (polyethylene terephthalate or polyethylene terephthalate glycol-modified) plastic (hereinafter PETG), the plastic sheets may be heated and then vacuum formed, or thermoformed on the model using a dental thermoforming machine, which adapts the sheet to the exact shape of the model. PET or other polymers or other such materials may also be used in addition to or instead of the PETG. According to a further aspect of the disclosure, one or more of the above-noted steps may be performed by a dentist in a dental office with or without the presence of the patient.

The thickness of the sheets after thermoforming may be approximately either 0.45 mm or 0.75 mm. According to an aspect of the disclosure, the thickness is uniform in all teeth. According to another of the disclosure, the thickness of the veneer varies according to the position of the veneer on the teeth. For example, the veneer may be thicker on less visible teeth, such as molars and premolar. For purposes of illustration, a rear portion of the veneer, which may have a key role in retaining the veneer in place, may encounter greater stresses and thus may be formed thicker. The thermoformed plastic sheets may then be removed from the model and sent to the design and finishing stage. It will be understood that the veneers may be ordered, molded, created, and delivered in other ways and using other or additional steps or materials.

Although the present invention has been described in relation to particular embodiments thereof, many other variations, combinations of features, modifications of features, and relations between structures will become apparent to those skilled in the art. Structures outlined as adjacent need not necessarily be positioned in that way, and not all steps or structures illustrated or described need necessarily be provided. Additional intervening steps or structures may be inserted. Structures described or illustrated as single units or structures may be substituted for by two or more structures, and structures described or illustrated as having multiple parts or divisions may be substituted for by a single structure. Uses and applications are described only by way of illustrative example.

Further, properties or features noted with respect to one part of a structure, or with respect to one aspect of the invention, may in addition or instead be present with respect to one or more other parts of the structure and/or one or more other aspects of the invention. Various properties or features described with respect to one aspect of the invention may be combined with one or more properties or features described with respect to another aspect of the invention. Many such combinations not explicitly noted are contemplated. It is preferred, therefore, that the present invention not be limited by the specific disclosure herein.
30 mold
40 dental veneer
41 cheek side of veneer
42 lingual side of veneer
42a lingual side ridge of veneer at the front
42b lingual side ridge of the veneer at the molars
46 molar cup interior
51 occlusal aperture
52 occlusal slot
61 occlusal hole
70 veneer with only one side

What is claimed is:

1. A removable dental veneer configured to secure onto a row of teeth of a patient, the dental veneer comprising:
a pair of rear portions configured to secure the dental veneer onto the row of teeth, each rear portion positioned at a distal end of the veneer and comprising:
a buccal side configured to cover at least a majority of a buccal surface of at least one rear tooth, an occlusal side configured to cover an occlusal surface of the at least one rear tooth, and a lingual side configured to cover at least a majority of a lingual surface of the at least one rear tooth; and
a front portion positioned between the pair of rear portions and comprising:
a buccal side configured to cover a buccal surface of each tooth of the row of teeth between the pair of rear portions, and an occlusal side configured to cover an occlusal surface of each tooth of the row of teeth between the pair of rear portions,
wherein the front portion is free of a surface that covers a majority of the lingual surface of any tooth of the row of teeth between the pair of rear portions.

2. The dental veneer of claim 1, wherein each rear portion is configured to cover at least two rearmost teeth or three rearmost teeth.

3. The dental veneer of claim 1, in combination with a corresponding removable dental veneer configured to secure onto an opposing row of the patient's teeth, the corresponding dental veneer comprising:
a corresponding pair of rear portions configured to secure the corresponding dental veneer onto the opposing row of teeth, each rear portion of the corresponding pair positioned at a distal end of the corresponding dental veneer and comprising:
a buccal side configured to cover substantially all of a buccal surface of at least one rear tooth, an occlusal side configured to cover substantially all of an occlusal surface of the at least one rear tooth, and a lingual side configured to cover substantially all of a lingual surface of the at least one rear tooth; and
a corresponding front portion positioned between the corresponding pair of rear portions and comprising:
a buccal side configured to cover substantially all of a buccal surface of each tooth of the opposing row of teeth between the corresponding pair of rear portions, and an occlusal side configured to cover substantially all of an occlusal surface of each tooth of the opposing row of teeth between the corresponding pair of rear portions,
wherein the corresponding front portion is free of a surface that covers a majority of the lingual surface of any tooth of the opposing row of teeth between the corresponding pair of rear portions,
wherein in the dental veneer, the buccal side of the front portion covers substantially all of the buccal surface of each tooth of the row of teeth between the pair of rear portions, and the occlusal side covers substantially all of the occlusal surface of each tooth of the row of teeth between the pair of rear portions.

4. The dental veneer of claim 1, wherein the pair of rear portions is configured to retain the dental veneer on the row of teeth by elastic deformation of the pair of rear portions and free of an adhesive compound.

5. The dental veneer of claim 1, wherein each rear portion is configured to cover an entirety of the occlusal surface of the at least one rear tooth.

6. The dental veneer of claim 1, wherein the front portion comprises:
a lingual ridge extending from the occlusal side and positioned over a lingual portion of the lingual surfaces immediately adjacent the occlusal surfaces of the row of teeth between the rear portions,
wherein the lingual ridge covers less than an tenth of the visible lingual surfaces of the row of teeth between the rear portions.

7. The dental veneer of claim 1, wherein the dental veneer is a customized dental veneer fitted for the patient's row of teeth.

8. The dental veneer of claim 1, wherein the dental veneer is one of translucent or semi-transparent.

9. The dental veneer of claim 1, wherein the dental veneer is made substantially entirely of at least one of polyethylene terephthalate or PETG.

10. A removable dental veneer configured to secure onto a row of teeth of a patient, the dental veneer comprising:
a pair of rear portions configured to secure the dental veneer onto the row of teeth, each rear portion positioned at a distal end of the dental veneer and comprising:
a buccal side configured to cover at least a majority of the buccal surface of at least one rear tooth of the row of teeth,
a lingual side configured to cover at least a majority of a lingual surface of the at least one rear tooth,
an occlusal side configured to cover a periphery of an occlusal surface of the at least one rear tooth and defining a slot extending from a distal end of the row of teeth over the occlusal surface of the at least one rear tooth, and the occlusal side comprising a ridge adjacent the slot and positioned adjacent a second surface of the at least one rear tooth, the second surface being a mesial surface opposite the distal end of the row of teeth, the ridge connecting the buccal side and the lingual side,
wherein the occlusal side is configured to cover a periphery of an occlusal surface of an additional rear tooth immediately adjacent the at least one rear tooth, the occlusal side defining an aperture exposing at least a majority of the occlusal surface of the additional rear tooth; and
a front portion positioned between the pair of rear portions and comprising:
a buccal side configured to cover a buccal surface of each tooth of the row of teeth between the pair of rear portions, and an occlusal side configured to cover an occlusal surface of each tooth of the row of teeth between the pair of rear portions,
wherein the front portion is free of a surface that covers a majority of the lingual surface of any tooth of the row of teeth between the pair of rear portions.

11. The dental veneer of claim 10, wherein the aperture exposes most of the occlusal surface of the additional tooth.

12. The dental veneer of claim 10, wherein an entirety of the perimeter of the slot is curvilinear and free of acute angles.

13. The dental veneer of claim 10, wherein the slot is positioned to expose at least a majority of the occlusal surface of the at least one tooth.

14. The dental veneer of claim 10, wherein the pair of rear portions is configured to retain the dental veneer on the row of teeth by elastic deformation of the pair of rear portions and free of an adhesive compound.

15. The dental veneer of claim 10, wherein the front portion comprises:
a thin lingual ridge extending from the occlusal side and positioned over a periphery of the lingual surfaces of the row of teeth immediately adjacent the occlusal surfaces of the row of teeth between the pair of rear portions.

16. The dental veneer of claim 10, wherein the dental veneer is a customized dental veneer fitted for the patient's row of teeth.

17. The dental veneer of claim 10, wherein the slot is configured to reveal an entirety of the occlusal surface of the at least one rear tooth except for a thin occlusal ridge at the buccal, lingual and mesial periphery of the occlusal surface.

18. The dental veneer of claim 10, wherein dental veneer is one of translucent or semi-transparent.

19. The dental veneer of claim 10, wherein the dental veneer is made substantially entirely of at least one of polyethylene terephthalate or PETG.

20. A removable dental veneer configured to secure onto a row of teeth of a patient, the dental veneer comprising:
a pair of rear portions configured to secure the dental veneer onto the row of teeth, each rear portion positioned at a distal end of the veneer and comprising:
a buccal side configured to cover at least a majority of a buccal surface of at least one rear tooth, a lingual side configured to cover at least a majority of a lingual surface of the at least one rear tooth, and an occlusal side configured to cover at least a majority of an occlusal surface of the at least one rear tooth, and the occlusal side defining a first aperture positioned to expose a central portion of the occlusal surface of the at least one tooth; and
a front portion positioned between the pair of rear portions and comprising:
a buccal side configured to cover a buccal surface of each tooth of the row of teeth between the pair of rear portions, and an occlusal side configured to cover an occlusal surface of each tooth of the row of teeth between the pair of rear portions.

21. The dental veneer of claim 20, wherein the front portion further comprises a lingual side configured to cover at least a majority of the lingual surface of each tooth of the row of teeth between the pair of rear portions.

22. The dental veneer of claim 20, wherein the front portion further comprises a slim lingual ridge configured to cover a portion of a lingual surface of each tooth of the row of teeth between the pair of rear portions,
wherein the slim lingual ridge covers no more than a tenth of the lingual surface of each tooth of the row of teeth between the pair of rear portions.

23. The dental veneer of claim 20, wherein the first aperture is sized and positioned to expose only a central portion of the occlusal surface of the at least one tooth and the occlusal side is configured to cover all remaining portions of the at least one tooth.

24. The dental veneer of claim 20, wherein the at least one tooth comprises a rearmost tooth and a second tooth adjacent the rearmost tooth, and the occlusal side of the rear portion defines a second aperture positioned to expose a central portion of the occlusal surface of the second tooth, the second aperture being of the same size and shape as the first aperture,
wherein the occlusal side of the rear portion is configured to cover all remaining portions of the occlusal side of the rearmost tooth and the occlusal side of the second tooth.

25. The dental veneer of claim 20, wherein the first aperture is substantially round.

26. The dental veneer of claim 20, wherein the first aperture has curvilinear edges.

27. The dental veneer of claim 20, wherein the at least one tooth comprises a rearmost tooth, a second tooth adjacent the rearmost tooth, and a third tooth adjacent the second tooth, and the occlusal side of the rear portion defines a second aperture positioned to expose a central portion of the occlusal surface of the second tooth and a third aperture positioned to expose, respectively, central portions of the occlusal surfaces of the second and third teeth, the second aperture and the third aperture being substantially of the same size and shape as the first aperture,
wherein the occlusal side of the rear portion is configured to cover all remaining portions of the occlusal surfaces of the rearmost tooth, the second tooth, and the third tooth.

28. The dental veneer of claim 27, wherein each of the first, second and third apertures exposes a minority of the occlusal surface of the respective tooth while a majority of the respective tooth is covered by the occlusal side of the veneer.

29. The dental veneer of claim 20, wherein the dental veneer is a customized dental veneer fitted for the patient's row of teeth.

30. The dental veneer of claim 20, wherein the dental veneer is one of translucent or semi-transparent.

31. The dental veneer of claim 20, wherein the dental veneer is made substantially entirely of at least one of polyethylene terephthalate or PETG.

32. A dental veneer configured to be secured onto a row of teeth of a patient, the dental veneer comprising:
a buccal side configured to cover a buccal surface of each tooth of the row of teeth; and
an occlusal side configured to cover an occlusal surface of each tooth of the row of teeth,
wherein the dental veneer is free of a surface that covers a majority of the lingual surface of any tooth of the row of teeth.

33. The dental veneer of claim 30, further comprising:
a slim lingual ridge extending from the occlusal side and positioned over a peripheral portion of the lingual surfaces of the row of teeth immediately adjacent the occlusal surfaces of the row of teeth.

34. The dental veneer of claim 30, wherein the buccal side covers substantially all of the buccal surface of each tooth of the row of teeth, and the occlusal side covers substantially all of the occlusal surface of each tooth of the row of teeth.

35. The dental veneer of claim 30, wherein the dental veneer is a customized dental veneer fitted for the patient's row of teeth and configured to be secured to the row of teeth by an adhesive compound.

36. The dental veneer of claim 30, wherein the dental veneer is one of translucent or semi-transparent.

37. The dental veneer of claim 30, wherein the dental veneer is made substantially entirely of at least one of polyethylene terephthalate or PETG.
